(19) United States
(12) Patent Application Publication
(10) Pub. No.: US 2020/0312457 A1
(43) Pub. Date: Oct. 1, 2020

(54) METHOD AND SYSTEM FOR CREATING SYNTHETIC UNSTRUCTURED FREE-TEXT MEDICAL DATA FOR TRAINING MACHINE LEARNING MODELS

(71) Applicant: The Trustees of Indiana University, Indianapolis, IN (US)

(72) Inventors: Suranga N. Kasthurirathne, Indianapolis, IN (US); Shaun J. Grannis, Indianapolis, IN (US)

(73) Assignee: The Trustees of Indiana University, Indianapolis, IN (US)

(21) Appl. No.: 16/831,971

(22) Filed: Mar. 27, 2020

Related U.S. Application Data
(60) Provisional application No. 62/825,243, filed on Mar. 28, 2019.

(51) Int. Cl. G16H 50/20; G06N 20/00; G06N 3/04; G16H 50/50

(52) U.S. Cl. CPC G16H 50/20; G16H 50/50; G06N 3/0454; G06N 20/00

(57) ABSTRACT

A method is provided for creating synthetic unstructured free-text medical data that closely mimics real data for enabling machine learning research, but with limited re-identification risk. The method includes leveraging two neural networks that compete with each other (adversarial networks) to create a synthetic message dataset that closely mimics the real medical data. Machine learning models trained using the synthetic data yield performance metrics that are statistically similar to models trained using the real dataset, ensuring that our approach can be used to replicate machine learning studies. Further, the synthetic message datasets can be easily shared with researchers with limited re-identification risk.

### RELATED APPLICATIONS

The present application claims the benefit of U.S. Provisional Application No. 62/825,243, filed Mar. 28, 2019, and entitled Method and System for Creating Synthetic Unstructured Free-Text Medical Data for Training Machine Learning Models, which is incorporated herein by reference.

### GOVERNMENT SUPPORT CLAUSE

None.

### TECHNICAL FIELD

The present disclosure relates generally to medical data processing systems and more particularly to methods and systems for creating synthetic unstructured free-text medical data.

### BACKGROUND

Rapid uptake of Health Information Systems (HIS) has enabled the accessibility and availability of structured and unstructured electronic health data. These data, together with the rapid evolution of Artificial Intelligence (AI) and various analytical and machine learning toolkits has led to the widespread development of machine learning solutions designed to address organizational-level challenges using organizational-level data. However, the current U.S. regulatory framework limits sharing of Patient Health Identifiers (PHI) outside the healthcare organization. Limited or burdensome data access hinders (a) sharing and re-using machine learning solutions across larger audiences, (b) promoting inter-organizational collaboration addressing various healthcare challenges, and (c) building generalized machine learning models targeting diverse populations.

Restrictions in sharing PHI limit cross-organizational re-use of free-text medical data. De-identification efforts focus on patient demographics removal, and may be vulnerable to re-identification based of clinical features. Generative Adversarial Networks (GAN) can be used to produce synthetic unstructured free-text medical data with low re-identification risk, and assess the suitability of using these datasets to replicate machine learning models.

There have been significant efforts to de-identify structured and unstructured patient data for research and dissemination purposes. Traditional de-identification efforts focus on the perturbation of potentially identifiable patient demographic attributes such as names, addresses, identifiers, and contact information via randomization, suppression or generalization. However, such efforts are not foolproof—patient records scrubbed of PHI may be susceptible to re-identification based on residual clinical information contained in symptoms, diagnosis, medications or lab results. This significantly impacts de-identification of structured data due to difficulty in identifying potentially sensitive information from free-text data.

Researchers have proposed various approaches for creating synthetic data that mimics clinical patterns in medical records as a solution to re-identification risk based on clinical information. A synthetic patient dataset that has been scrubbed of any PHI elements using traditional de-identification methods would be significantly harder to re-identify than a real dataset that has only been scrubbed of PHI elements. However, previous synthetic data generation efforts have resulted in data that are not sufficiently realistic for machine learning tasks.

As such, there is a need to develop an enhanced medical data processing system providing an improved electronic health data for the health systems.

### SUMMARY

The present disclosure provides methods and systems for training a series of GAN models using unstructured free-text laboratory messages pertaining to salmonella, and identified the most accurate models for creating synthetic datasets that reflect the informational characteristics of the original test data. Similarity of the synthetic data is accessed by evaluating the Natural Language Generation (NLG) metrics that compare the real and synthetic datasets, using the Random Forest classification algorithm to train decision models capable of identifying salmonella cases using the top 5, 10, 15 and 20 features extracted from the real and synthetic datasets, and testing a holdout set of laboratory messages. These models are compared using sensitivity, specificity, F1-measure and area under the receiver operating characteristic (ROC) curve values.

Natural Language Generation (NLG) metrics comparing the real and synthetic datasets demonstrated a high degree of similarity. Decision models generated using these datasets reported high performance metrics. Additionally, there was no statistically significant difference in performance measures reported by models trained using real and synthetic datasets.

The results inform two challenges; the use of GAN models to generate synthetic unstructured free-text data with limited re-identification risk, and use of this data to enable collaborative research and re-use of machine learning models.

In one embodiment of the present disclosure, a method of processing real medical data is disclosed. The method includes leveraging two neural networks (e.g., adversarial networks) that compete with each other to create a synthetic message dataset that closely mimics the real medical data. The synthetic message dataset having synthetic medical data being substantially indistinguishable from the real medical data, and lowering re-identification risk based on matches between a synthetic data record in the synthetic medical data and a real data record in the real medical data. Machine learning models trained using the synthetic data yield performance metrics that are statistically similar to models trained using the real dataset, ensuring that our approach can be used to replicate machine learning studies. Further, the synthetic message datasets can be easily shared with researchers with limited re-identification risk.

In one example, generating the first message comprises generating a positive train dataset of the first message dataset for training the first adversarial network. In a variation, generating the first message comprises generating a positive holdout dataset of the first message dataset that excludes the positive train dataset.

In another example, generating the second message comprises generating a negative train dataset of the second message dataset for training the second adversarial network. In a variation, generating the second message comprises generating a negative holdout dataset of the first message dataset that excludes the negative train dataset.

Positive and negative train datasets are merged, and top features identified using Gini impurity. The top 5, 10, 15 and 20 features are identified, and used to train a series of Random Forest decision models capable of predicting positive or negative cases of Salmonella. These models are tested using the (a) positive and negative holdout datasets, and (b) real holdout datasets. Model performance is compared using various performance measures.

Assessing re-identification risk associated with the synthetic message dataset involves a presence disclosure test where hamming distances between the synthetic data record and the real data record are calculated. In a variation, the method further includes determining a degree of the re-identification risk based on the hamming distance threshold.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

The above-mentioned and other features of this disclosure and the manner of obtaining them will become more apparent and the disclosure itself will be better understood by reference to the following description of embodiments of the present disclosure taken in conjunction with the accompanying drawings, wherein:

FIG. 1 illustrates a workflow of a medical data processing system depicting a data processing process from a laboratory message extraction to a decision model evaluation in accordance with embodiments of the present disclosure;

FIG. 2 illustrates plots depicting the mean, median and 95% confidence intervals of the top 7 features that co-occurred across salmonella positive reports in the test and synthetic datasets;

FIG. 3 illustrates the sensitivity, specificity, F1-measure and Area under the ROC curve scores reported by decision models built using the top 5, 10, 15 and 20 real and synthetic features upon being tested using the holdout test datasets; and

FIG. 4A illustrates a frequency of positive (synthetic) reports matched with positive (train) reports (hamming threshold <=10);

FIG. 4B illustrates a frequency of negative (synthetic) reports matched with negative (train) reports irrespective of report status (hamming threshold <=10);

FIG. 4C illustrates a frequency of positive (synthetic) reports matched with positive (train) reports (hamming threshold <=20);

FIG. 4D illustrates a frequency of negative (synthetic) reports matched with negative (train) reports irrespective of report status (hamming threshold <=20); and

FIG. 5 illustrates a variance of gini impurity scores reported by the top 50 features extracted from the real and synthetic datasets.

While the present disclosure is amenable to various modifications and alternative forms, specific embodiments have been shown by way of example in the drawings and are described in detail below. The intention, however, is not to limit the present disclosure to the particular embodiments described. On the contrary, the present disclosure is intended to cover all modifications, equivalents, and alternatives falling within the scope of the present disclosure as defined by the appended claims.

### DETAILED DESCRIPTION

The embodiments disclosed below are not intended to be exhaustive or to limit the invention to the precise forms disclosed in the following detailed description. Rather, the embodiments are chosen and described so that others skilled in the art may utilize their teachings.

### INTRODUCTION

Generative Adversarial Networks (GAN) are a class of deep learning algorithms that offer significant promise to improve synthetic data generation. GAN algorithms are implemented by a system of two neural networks. One neural network, the generator, attempts to create synthetic data, while the other neural network, the discriminator, seeks to distinguish between synthetic data and real data. As these networks are trained, the generator network successfully develops synthetic data that cannot be flagged by the discriminator. Initial GAN models were designed to mimic real-valued data. As such, they have been used to produce high quality categorical and image datasets. In the healthcare domain, GAN models have been used to generate numerical clinical data that is statistically similar to real data.

Recent improvements to GAN algorithms enable them to generate synthetic free-text data. Researchers have applied these models to successfully generate text data such as molecules encoded as text sequences, musical melodies, reviews, dialogues, poetry and image captions. These innovations offer much potential to the medical field, where a large quantity of clinical information may be trapped within unstructured free-text.

Materials and Methods

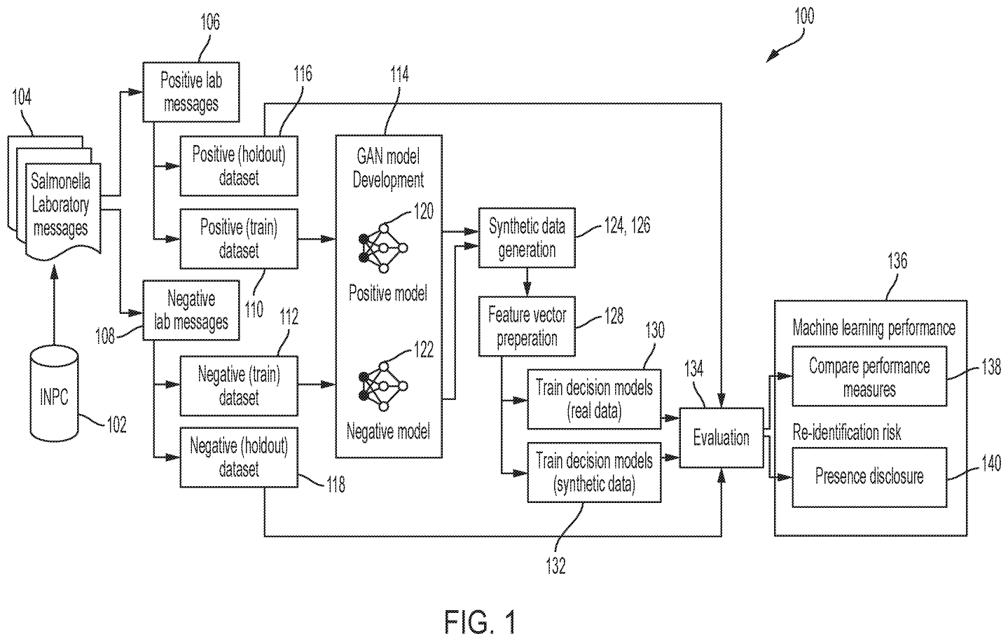

FIG. 1 shows an exemplary workflow of a medical data processing system 100 from data extraction to decision model evaluation. Detailed descriptions of medical data processing system 100 are provided below.

Test Data Selection

All laboratory messages are extracted pertaining to cases of Salmonella reported to the Indiana Network for Patient Care (INPC) 102 during 2016-2017. The INPC is a statewide Health Information Exchange (HIE) that facilitates interoperability across 117 hospitals, 38 health systems, other free-standing laboratories, and physician practices across the state of Indiana. These messages, which were obtained in the form of Health Level Seven (HL7) version 2 messages, are parsed and extracted the free-text report data included in each message. Laboratory messages for salmonella 104 were selected due to the semi-structured nature of the HL7 messages, which allowed us to separate PHI from the unstructured text, as well as the brevity of the free-text laboratory messages. Each message was manually reviewed, and labelled as positive 106 or negative 108 for Salmonella. Approximately 90% of each of the positive and negative salmonella messages are randomly selected, hereafter known as positive (train) 110 and negative (train) 112 datasets for training GAN models 114. The remainder of the datasets, hereafter known as the positive (holdout) 116 and negative (holdout) 118 datasets, were used to test the performance of GAN generated data.

Development of GAN Models for Synthetic Data Generation

In one embodiment, SeqGAN, a GAN algorithm designed to generate textual data, is used. SeqGAN models approach the sequence generation procedure as a sequential decision-making process. The generative model is treated as an agent of reinforcement learning; the state is the generated tokens while the action is the next token to be generated. The discriminator evaluates the sequence and feeds back the evaluation to guide the learning of the generative model.

GAN models consist of a number of parameters that can be fine-tuned to optimize model performance. Model performance is explored by training multiple GAN models, e.g., a positive GAN model 120 generated based on the positive (train) 110 dataset and a negative GAN model 122 generated based on the negative (train) 112 dataset, and varying several parameters (Appendix A). A Gaussian distribution can be adopted as the default initial parameter for all generators. Performance of these models were compared using two document similarity based metrics; embedding similarity, which measures similarity between two documents as the performance measure 138 to evaluate GAN models, and Negative Log Likelihood (NLL)-test, which evaluates a model's capacity to fit real test data. Optimal models selected using this approach were used to generate positive (synthetic) 124 and negative (synthetic) 126 laboratory messages. To build compatible decision models, n positive synthetic reports are generated, where n equals the number of positive (train) messages, and m negative synthetic reports, where m equals the number of negative (train) messages. SeqGAN models are trained using Texygen, a benchmarking platform for GAN based text generation models.

Machine Learning Process 136

Features extracted from the positive (train) 110 and negative (train) 112 datasets, (jointly known as the real dataset), and positive (synthetic) 124 and negative (synthetic) 126 datasets (jointly known as the synthetic dataset) were used to train multiple classification models using the following approach.

Feature Extraction

The feature extraction process 128 can be mimicked and adopted in a previous work on predicting cancer cases using free-text data obtained from the INPC. A Perl script can be developed to parse the positive and negative training datasets, and identify all unique stemmed tokens present within these reports. Next, the Negex algorithm is used to identify the context of use (positive or negative) for each stem. The presence of each feature in positive and negated context is counted, and used this data to prepare an input vector for each laboratory message. A similar approach was used to generate vectors of counts representing each message in the synthetic dataset. Decision model building and evaluation

The Gini impurity metric is applied to rank features in the real and synthetic datasets by order of importance. The subsets of the top 5, 10, 15 and 20 features are used and selected from the real and synthetic datasets to train a series of decision models 130, 132 using the Random Forest classification algorithm, e.g., a decision model 130 using the real datasets and a decision model 132 using the synthetic dataset. Random forest was selected due to its proven track record in health care decision-making applications. The real and synthetic decision models were tested using feature vectors derived from the positive and negative holdout datasets. Sensitivity (True Positive Rate or Recall), specificity (True Negative Rate), F1-Measure and Area Under the ROC Curve (AUC) for each decision model are calculated. Paired t-tests were used to compare the performance of decision models trained using the real or synthetic datasets.

Evaluation 134 of Re-Identification Risk

Risk of presence disclosure 140, aka membership inference assesses an attackers' ability to determine if any real patient records in their possession were used to train GAN models by comparing these records against the synthetic patient dataset. Assessing risk of presence disclosure 140 ensures the privacy of individuals whose data was used to train a decision model, as well as the interests of the healthcare entity where the individual received treatment. Thus, presence disclosure 140 is a widely evaluated measure of re-identification risk. The risk of presence disclosure 140 using the following experiment is assessed, and re-purposed vectors are also assessed that represented the training and synthetic datasets using binary values representing the presence or absence of each feature in positive and negative context. Each synthetic record is compared with all training messages using various hamming distance cutoff scores, a measure of the minimum number of substitutions required to change one string into the other. The identification of a synthetic record that matched with any training message using a hamming distance equal or smaller to the hamming score threshold would label it as a ‘match’ to the synthetic record under study. The frequency of matches is computed across each hamming score threshold, and these metrics are used to evaluate re-identification risk.

Results

A total of 6,770 laboratory messages are identified pertaining to salmonella. Manual review labelled 1,213 (17.91%) of these messages as positive, and 5,557 (82.08%) as negative. The optimal SeqGAN models are identified for generating positive and negative laboratory messages using hyperparameters identified in appendix A. Using these models, 1092 positive synthetic messages and 5001 negative synthetic messages are generated to correspond with the 90% training messages for each dataset. Appendix B presents representative samples from the positive (train), negative (train), positive (synthetic) and negative (synthetic) lab report sets. These samples were manually reviewed, and any PHI elements masked. As seen in appendix B, the only PHI elements identified within these report sets were date, time and report identifier fields.

Several Natural Language Generation (NLG) measures are computed to evaluate similarity between real and synthetic datasets; a) Bilingual Evaluation Understudy (BLEU) scores are widely used to compare similarity between real and synthetic datasets. BLEU-1, BLEU-2, BLEU-3 and BLEU-4 scores are calculated that evaluated the quality of synthetic datasets using 1-gram, 2-gram, 3-gram and 4-gram matches respectively. b) Google-BLEU (GLEU) scores, a measure that seeks to address limitations in BLEU score calculations and are better suited for sentence level comparisons. The GLEU score is a composite of all 1-grams, 2-grams, 3-grams and 4-gram matches (table 1).

TABLE 1
Comparison of real and synthetic datasets using
various NLG measures.

| | Positive (train) vs. | Negative (train) vs. |
|---|---|---|
| NLG measure | Positive (synthetic) | Negative (synthetic) |
| BLEU-1 | 0.913 | 0.944 |
| BLEU-2 | 0.675 | 0.742 |
| BLEU-3 | 0.480 | 0.552 |
| BLEU-4 | 0.331 | 0.409 |
| Google-BLEU | 0.249 | 0.328 |

These results indicate significant similarity between real and synthetic datasets. Further, NLG measures comparing negative (train) and negative (synthetic) datasets were higher than the positive (train) and positive (synthetic) datasets. Some negative reports may tend to have a higher similarity due to uniform text documenting negative status.

The positive (train) dataset comprised of 2,551 unique stemmed features. 1,827 (71.6%) of these features were present within the positive (synthetic) dataset. The negative (train) dataset comprised of 5,803 unique stemmed features. 4,093 (70.5%) of these stemmed features were present within the negative (synthetic) dataset. With stop words and dates removed, the overall training dataset of positive and negative reports consisted of 3810 unique stemmed features. 2651 (69.6%) of these were present within the overall synthetic dataset. Appendix C lists the top 20 features identified across the real and synthetic datasets using gini impurity scores. Appendix D presents the overlap between the top 5, 10, 15, 20, 50 and 100 features identified across the real and synthetic datasets. Significant similarity is noted between real and synthetic datasets with between 70% to 80% overlap across each of the feature subsets being compared.

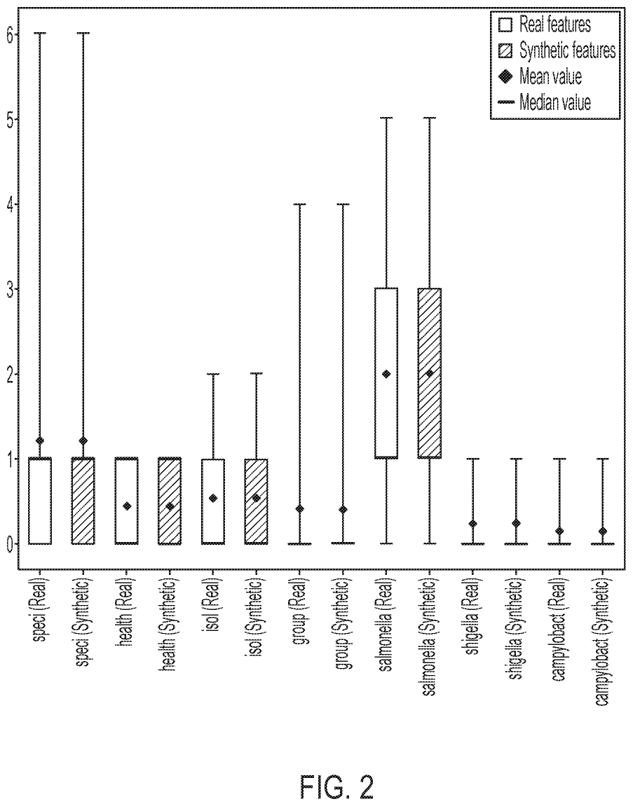

To evaluate if the synthetic feature sets generated by GAN models could accurately reflect positive/negated characteristics of features included in the training dataset, box whisker plots are developed depicting the mean, median and 95% confidence intervals of the top 7 features that co-occurred across the positive (train) and positive (synthetic) datasets (FIG. 2). Results suggest that the distribution of features are similar, with only slight variations.

FIG. 2 shows box whisker plots depicting the mean, median and 95% confidence intervals of the top 7 features that co-occurred across salmonella positive reports in the test and synthetic datasets.

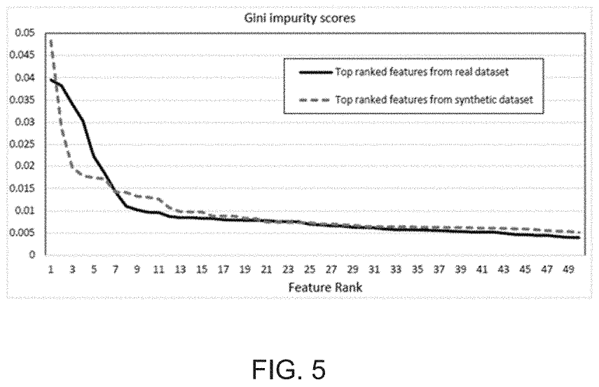

FIG. 5 shows the variation of gini impurity scores across the top 50 real and synthetic features. It is noted that gini impurity scores follow a similar trend across both feature sets. Variance between gini scores of each dataset are reduced even further beyond the top 20 features.

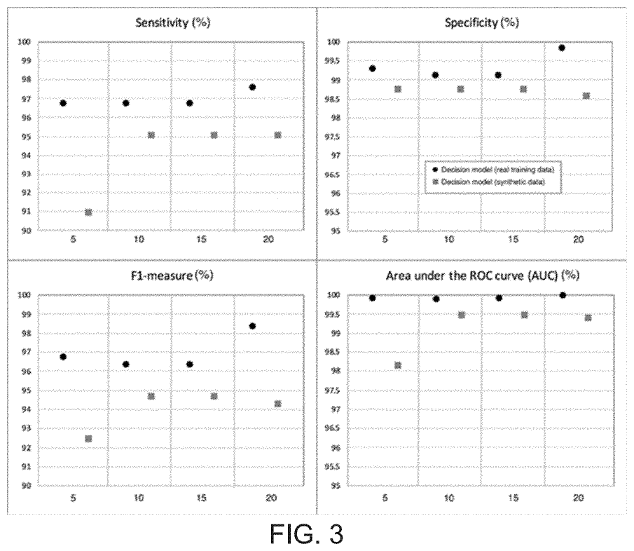

FIG. 3 shows the sensitivity, specificity, F1-measure and Area under the ROC curve scores reported by decision models built using the top 5, 10, 15 and 20 real and synthetic features upon being tested using the holdout test datasets.

Due to the discriminatory power of the features, each model achieved high-performance measures despite being trained on a small number of features. Further, paired t-tests indicated no significant difference between performance measures reported by real and synthetic decision models built using any of the feature subset sizes. Given high overlap between top 50 and 100 feature sets (appendix D), and the similarity of gini impurity scores reported by the top 50 features (FIG. 5) Decision models built using the top 50 and 100 real and synthetic features may also report statistically similar performance measures.

Evaluation of Re-Identification Risk

Results of the presence disclosure test are presented in appendix F. It can be concluded that these results indicate acceptable levels of re-identification risk given that the number of positive matches identified across hamming thresholds of 10 and 20 were reasonably small.

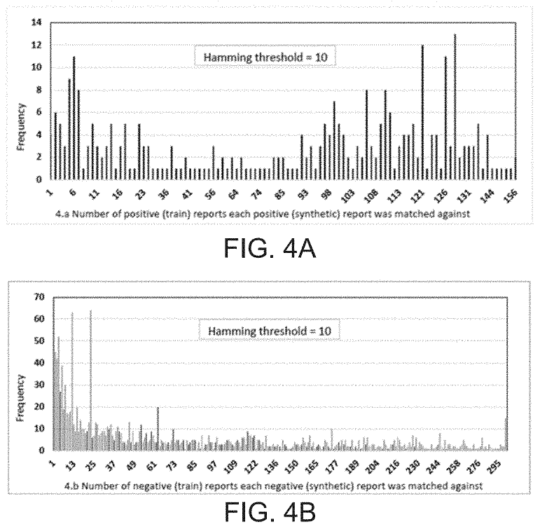

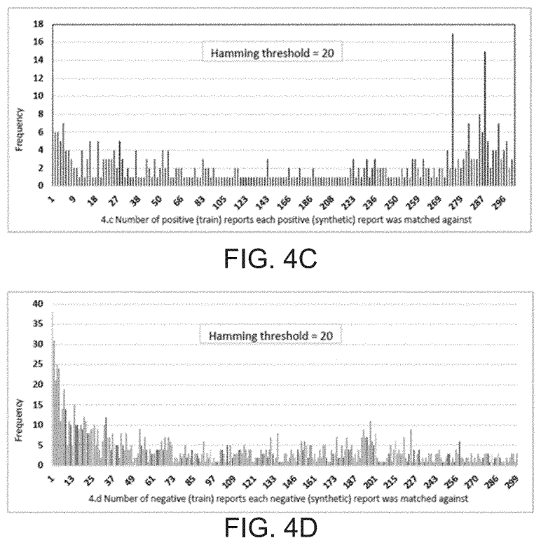

FIG. 4A shows a frequency of positive (synthetic) reports matched with positive (train) reports (hamming threshold <=10). FIG. 4B shows a frequency of negative (synthetic) reports matched with negative (train) reports irrespective of report status (hamming threshold <=10). FIG. 4C shows a frequency of positive (synthetic) reports matched with positive (train) reports (hamming threshold <=20). FIG. 4D shows a frequency of negative (synthetic) reports matched with negative (train) reports irrespective of report status (hamming threshold <=20).

It can be determined that re-identification risk is greater when,

a) A synthetic report is matched with a smaller number of real reports. Linking a synthetic report to a smaller number of real reports offer attackers a greater chance of pinpointing true matches via manual review. Re-identification risk falls as the number of real reports matched with a single synthetic report increases, as attackers must manually review each of these matches to pinpoint patients.

b) Synthetic reports are matched with real reports using smaller hamming cutoff thresholds. Smaller hamming distance thresholds indicate smaller differences between records, and thus, raises the likelihood that two matched reports are the same.

An evaluation of matches across hamming distances of 10 and 20 presents that positive synthetic reports were matched to positive real reports (FIGS. 4.a and 4.c) at a lower rate than negative reports (FIGS. 4.b and 4.d). As such, they poise significantly low chance of re-identification. It can be hypothesized that negative reports were matched with more certainty because they consisted of uniform text documenting negative status. As anticipated, chances of matching a negative synthetic and real reports were larger than matching positive synthetic and real reports.

Discussion

The results further two challenges; the use of GAN models to generate synthetic free-text medical data with limited re-identification risk, and use of these datasets to develop machine learning models with statistically similar performance metrics to models developed using the original test data, thereby enabling cross-institutional collaboration and broader dissemination of machine learning models.

Comparison of unique features across test and synthetic datasets revealed that the synthetic dataset contained only 69.6% of the features in the test dataset. This can be attributed to the mode collapse problem, which leads to reduced diversity of synthetic data. NLG scores reported by the models were compatible to scores reported by other efforts to generate synthetic text data extracted from non-medical sources. However, it is noted that the synthetic data presented reduced syntactic/grammatical correctness (appendix B), a common pitfall in deep learning based text generation approaches. However, this was irrelevant for the purposes as it is only sought to demonstrate that synthetic data could be used to replicate machine learning performance, and not as a tool for training or teaching of humans. Thus, no human evaluation of the synthetic reports was performed. The synthetic dataset also contained a quantity of recurring phrases such as hospital and laboratory test names. The recurrence of such phrases may have positively influenced NLG scores. Despite these limitations, there was 70-80% overlap between the top 5, 10, 15, 20, 50 and 100 features extracted from both datasets (appendix D). Performance measures generated by models trained using top 5, 10, 15 and 20 features extracted from the test and synthetic datasets were high, as well as statistically similar. These results present the possibility of using synthetic datasets to share machine learning solutions, and foster cross-institutional collaboration on various challenges.

The findings help inform data de-identification efforts. As discussed previously, de-identification efforts involve (a) removal of PHI elements, and (b) addressing re-identification risk based on clinical information in patient records. Adoption of GAN models alone do not result in de-identified data. However, synthetic data generation reduces re-identification risk by creating new patient records with similar, but different content. It also removes any 1-to-1 mapping between test and synthetic reports. The results using presence disclosure tests confirmed that synthetic datasets pose a small chance of re-identification based on clinical information. However, synthetic data produced by these efforts must undergo rigorous de-identification of PHI elements before they can be distributed for public use. Removal of PHI elements will not impact decision model performance as the top 100 features listed in appendix D did not include any PHI elements.

An alternate approach to evaluate re-identification risk is attribute disclosure, which evaluates an attackers' ability to derive additional attributes (features) for a patient based on a subset of attributes they are aware of. The datasets for attribute disclosure may not be evaluated as a) unlike longitudinal patient datasets that consist of varied clinical diagnoses that are not necessarily related, the salmonella lab reports consisted of very specific features that are often highly correlated. Thus, it would be relatively easy to predict missing features in the dataset based on those present. Secondly, a considerable number of features presented very low prevalence across the laboratory reports. Thus, predicting ‘absence’ of these features was relatively easy. However, these factors pose low risk to the patient because unlike studies that deal with clinical diagnosis that if revealed, may impact the patient's privacy, the dataset focuses on salmonella alone. As an example, discovery of any of the top features listed in appendix C using an attribute disclosure attack would not lead to any harm beyond the awareness that the patient was tested for, and diagnosed as positive or negative for Salmonella. In contrast, attribute disclosure across a different dataset may lead to discovery of multiple clinical diagnosis, patient demographics or other treatment information.

The following hypothetical scenario is proposed to demonstrate how the approach could be applied in a real-life setting; An organization that possesses rich free-text data sources, but lacks adequate machine learning expertise can leverage the approach to create synthetic data. They de-identify and share the synthetic data with experts who use it to build machine learning models. Once optimal models have been identified, they can be implemented across the original dataset with compatible performance measures.

The test dataset consisted of structurally similar reports describing a very specific illness. This, together with the overall simplicity of the predictive outcomes (positive vs. negative for salmonella) may have contributed to the positive results. Datasets that are not structurally similar, nor restricted to a specific illness, or consist of more colloquial language may be harder mimic, and thus, produce less optimal results. Such datasets may require more robust decision models built using other free-text friendly GAN models such as Maximum-Likelihood augmented discrete Generative Adversarial Networks (MaliGAN) or Long Text Generative Adversarial Networks (LeakGAN), and more complex feature vectors consisting of n-grams. Further, the approach was restricted to mimicking synthetic free-text data. The models can learn or mimic the significance of various numeric values such as age or other measurements present in free-text data. This feature may not impact the performance of the current effort as no numerical values were selected as top features. However, it may impact models built using other datasets.

Future research avenues include use of GAN models to create truly de-identified synthetic free-text data that does not require additional de-identification, and expansion of the work across other more challenging healthcare datasets. Further, other researchers have demonstrated the ability to mimic numerical and categorical patient data using GAN models. Integrating these efforts would enable researchers to share comprehensive synthetic patient health records consisting of both structured and unstructured data for secondary research purposes.

### CONCLUSION

GAN models can be used to generate synthetic unstructured free-text medical data that can be used to replicate the performance of machine learning models with high, as well statistically similar results. Further, synthetic datasets poise limited risk of re-identification based on clinical features. As such, these synthetic datasets can be easily de-identified, and used to champion cross-organizational collaboration efforts.

The above detailed description and the examples described therein have been presented for the purposes of illustration and description only and not for limitation. For example, the operations described can be done in any suitable manner. The methods can be performed in any suitable order while still providing the described operation and results. It is therefore contemplated that the present embodiments cover any and all modifications, variations, or equivalents that fall within the scope of the basic underlying principles disclosed above and claimed herein.

Embodiments of the present disclosure are described by way of example only, with reference to the accompanying drawings. Further, the following description is merely illustrative in nature and is in no way intended to limit the disclosure, its application, or uses. As used herein, the term “unit” or “module” refers to, be part of, or include an Application Specific Integrated Circuit (ASIC), an electronic circuit, a processor or microprocessor (shared, dedicated, or group) and/or memory (shared, dedicated, or group) that executes one or more software or firmware programs, a combinational logic circuit, and/or other suitable components that provide the described functionality. Thus, while this disclosure includes particular examples and arrangements of the units, the scope of the present system should not be so limited since other modifications will become apparent to the skilled practitioner.

Furthermore, while the above description describes hardware in the form of a processor executing code, hardware in the form of a state machine, or dedicated logic capable of producing the same effect, other structures are also contemplated. Each unit or component can be operated as a separate unit, and other suitable combinations of sub-units are contemplated to suit different applications. Also, although the units are illustratively depicted as separate units, the functions and capabilities of each unit can be implemented, combined, and used in conjunction with/into any unit or any combination of units to suit different applications.

It is to be understood that the above description is intended to be illustrative, and not restrictive. Many other embodiments will be apparent to those of skill in the art upon reading and understanding the above description. For example, it is contemplated that features described in association with one embodiment are optionally employed in addition or as an alternative to features described in associate with another embodiment. The scope of the present disclosure should, therefore, be determined with reference to the appended claims, along with the full scope of equivalents to which such claims are entitled.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

### APPENDICES

APPENDIX A
List of hyperparameters evaluated as part of the SeqGAN
training process.

| Parameter | Description | Variations attempted |
|---|---|---|
| Pre-training | The generator is trained for n epochs, | Increments of 5 |
| epochs | followed by n epochs for the | between 10 and 100 |
| Adversarial | Number of adversarial epochs | Increments of 5 |
| epochs | | between the values |
| | | 5 and 50 |
| Embedding | Dimensionality of embedding layer | 32, 64, 128 |
| dimensions | | |
| Hidden | Number of neurons in hidden layer | 32, 64, 128 |
| dimensions | | |
| sequence | Length of each training sequence | Increments of 10 |
| length | | between the values |
| | | 10 and 120 |

Appendix B. Representative samples of the train and synthetic datasets with HL7 tags removed. Any potential PHI elements within these reports were replaced with <tags>.

Positive (Train) Messages

a) culture in progress. identifications performed by maldi tof mass spectrometry were developed and performance characteristics were determined an organization. salmonella species numerous. susceptibility not routinely performed. gastroenteritis due to non typhoidal salmonella spp. is generally self limiting in patients without underlying medical issues. for Salmonella typhi isolates azithromycin is the drug of choice. identified by maldi tof mass spectrometry. sent to a state department of health.

b) additional organisms present as probable contaminants. salmonella species 100000 cfu/ml this strain tested resistant to naladixic acid. treatment of extra intestinal salmonella infections may not be eradicated by fluoroquinolone treatment. therefore ciprofloxacin and levofloxacin are reported as resistant.

c) no Shigella aeromonas Plesiomonas edwardsiella or campylobacter isolated. no predominant growth of Klebsiella oxytoca present. identifications performed by maldi tof mass spectrometry were developed and performance characteristics were determined by an organization. salmonella species numerous. susceptibility not routinely performed. gastroenteritis due to non typhoidal salmonella spp. is generally self limiting in patients without underlying medical issues. for Salmonella typhi isolates azithromycin is the drug of choice. identified by maldi tof mass spectrometry. salmonella serotype Salmonella braenderup test performed by a state department of public health.

d) gram stain of blood culture vial indicates presence of gram negative bacillus. direct mass spectrometry testing performed on positive blood culture bottle indicates presence of salmonella species. additional testing including susceptibility when appropriate to follow. identifications performed by maldi tof mass spectrometry were developed and performance characteristics were determined by the organization. salmonella species identified by maldi tof mass spectrometry. sent to a state department of public health.

e) gram stain of blood culture vial indicates presence of gram negative bacillus. culture in progress. identifications performed by maldi tof mass spectrometry were developed and performance characteristics were determined by an organization. salmonella species identified by maldi tof mass spectrometry.

f) many salmonella species reportable disease moderate Enterococcus faecalis

g) accession <accession_number> final reports reportable disease positive for campylobacter antigen by enzyme immunoassay. negative for shiga toxin 1 or 2 by enzyme immunoassay. fecal specimens are routinely cultured for the most common enteric pathogens salmonella and shigella also included are the less common miscellaneous entericpathogens Aeromonas plesiomonas Edwardsiella vibrio and yersinia. two specimens are usually sufficient for feces culture since they yield 99% of the pathogens.

Negative (Train) Message

a) identifications performed by maldi tof mass spectrometry were developed and performance characteristics were determined by an organization. no Salmonella shigella Aeromonas plesiomonas edwardsiella isolated. no predominant growth of Klebsiella oxytoca present. one or more organisms were isolated and found to be normal flora through maldi tof mass spectrometry. Campylobacter jejuni numerous. identified by maldi tof mass spectrometry. drugs of choice are ciprofloxicin erythromycin clindamycin tetracycline.

b) one or more organisms were isolated and found to be normal flora through definitive biochemical testing. no Salmonella shigella Plesiomonas edwardsiella or campylobacter isolated. no predominant growth of Klebsiella oxytoca present. aeromonas species moderate. susceptibility not routinely performed. aeromonas spp. are associated with gastrointestinal disease. Symptoms are usually mild and self limiting. individuals with impaired immune systems or underlying malignancy are susceptible to more severe infection. antibiotics maybe indicated if symptoms are prolonged and in system is infections. identified by maldi tof mass spectrometry.

c) test culture stool specimen source rectum specimen type stool specimen date <date> <time> result date <date> <time> result status final result resulting at 111 Main St. culture shiga toxin negative no salmonella species no shigella species isolated no Escherichia coli o157 h7 isolated no Campylobacter jejuni no salmonella or shigella isolated normal enteric flora not present. report comment one of two test performed at a laboratory on 111 Main St. with John Smith.

d) order #<order_number> ordered by jeremy fisk source stool collected <date> <time> antibiotics at coll. received <date> <time> culture stool final <date> <time> <date> no salmonella species shigella species or E. coli 0157 h7 isolated <date> negative antigen screen for shiga toxins 1 and 2 produced by Escherichia coli [stec] negative antigen screen for campylobacter species

e) normal gi flora present preliminary report <date> at <time> no enteric pathogens isolated stool screened for Salmonella shigella Staphylococcus aureus campylobacter and sorbitol negative E. coli final report <date> at <time>

f) shiga toxin 1 and shiga toxin 2 absent no Campylobacter salmonella shigella E. coli o157 plesiomonas orvibrio isolated copy of report sent to infections control testing for Clostridium difficile toxin is recommended in lieu of culture or ova and parasite exam if the patient has been hospitilized for three or more days. if further testing is desired please collect a fresh stool specimen and order a Clostridium difficile antigen test. Aeromonas hydrophila group moderate infections are usually self limiting when isolated from feces only.

g) refer to micro report in meditech culture stool final no salmonella or shigella isolated negative for campylobacter by enzyme immunoassay negative for shiga toxin 1 and 2 by enzyme immunoassay this is a corrected result. a prior result that was reported as final has been changed. C. difficile toxin gene naat final positive test performed by nucleic acid amplification colonization rates of up to 50% have been reported in infants. a high ratio has also been reported in cystic fibrosis patients. end of report tests performed at the main laboratory 1000 E. State St.

Positive (Synthetic) Messages

a) client services present. moderate salmonella spp result progress called faxed to dr Jane Smith at <time> on <date> by dr John Doe dr Smith's office salmonella species sent to state lab salmonella group ser. jg salmonella spp

b) identifications performed by maldi tof mass spectrometry were developed and performance characteristics were determined by the organization. salmonella species identified by maldi tof mass spectrometry. salmonella species sent to indiana by Jane Doe [317. E. coli o157 no campylobacter isolated sent to a state department of health reportable confirmed by the state dept. of further testing performed. salmonella performed on up please contact the laboratory if serotyping is required.

c) 375 greater than 100000 cfu/ml salmonella species sent to indiana state lab staphylococcus spp susceptibility testing in progress performed by maldi tof mass spectrometry were developed and performance characteristics were determined by an organization. salmonella species sent to state department of health laboratory. salmonella species moderate. salmonella species reaction ampicillin 8 an infection. identification. disclaimer salmonella species sqxsalsp salmonella i ser. state lab performed. salmonella group b

d) salmonella spp chslb 372342007 salmonella sct salmonella spp salmonella spp susceptibility testing in progress called to 69687 [m3nim] by dr Jane's office Shigella campylobacter or organisms present as 2 to verified a cefazolin results cefazolin considered in patients without underlying medical issues. for Salmonella typhi isolates azithromycin is the drug of choice. sent to a state department of health laboratories

e) salmonella species isolated sent to a state department of health reportable organism will be sent to a state department of refer to Mr. John Smith

f) few salmonella no E. coli o157 no campylobacter isolated sent to a state department of report sent to the state department of health other confirmed by the state dept. salmonella serotype salmonella ser. oranienburg not performed. identification no further testing performed is desired please collect a fresh stool specimen and 64 amp/sulbac on <date> salmonella Kiambu

g) specimen sources tool final report salmonella group b salmonella serotype salmonella ser. enteritidis disclaimer salmonella sp.

Negative (Synthetic) Messages

a) test culture stool specimen source rectum specimen type stool specimen date <date> <time> result date <date> culture <time> result status final result resulting lab in lab at 5 Main St. culture result abnormal yes resulting lab esk main in lab at 5 Main St. end of yeast many shiga toxin negative no Campylobacter jejunino salmonella species shigella species or shigella isolated mrsa shigella species isolated. Escherichia coli o157 no

b) no salmonella no Shigella aeromonas Plesiomonas edwardsiella or campylobacter isolated. no predominant growth of Klebsiella oxytoca present.

c) see report stool culture preliminary report no Salmonella shigella or yersinia isolated by enzyme immunoassay. aeromonas spp. this is with erythromycin America clinical reported as final has been reported in cystic fibrosis patients. stool occult blood final end of report tests performed at main laboratory 50 St. State.

d) Staphylococcus aureus no Salmonella shigella Campylobacter yersinia isolated by enzyme immunoassay. performing locations p1 this culture. was performed at tmf central lab clia #15d0357169 35 Main St.

e) normal gi flora present no enteric pathogens isolated stool screened for Salmonella shigella Staphylococcus aureus campylobacter and sorbitol negative E. coli o157 this culture is a prior result no further 15d0662599 date called to difficile rn at dr. Joe's office on <date> at by Jill

f) test culture stool specimen type stool specimen received <date> <time> est final reports verified date/time <date> <time> final reports verified date/time <date> <time> no Salmonella shigella species isolated no salmonella species no shigella species isolated no salmonella or Shigella plesiomonas isolated. no Shigella aeromonas Plesiomonas edwardsiella or campylobacter isolated not routinely cultured is desired. no campylobacter species called to a department of at <time>

g) normal gi flora present no pathogens isolated stool screened for Salmonella shigella Staphylococcus aureus campylobacter and sorbitol negative E. coli o157 Aeromonas plesiomonas orvibrio isolated report comment fasting unknown test performed on this isolate. no Campylobacter jejuni Shigella sonnei sct be. warranted. n lafayette specimen and order a positive normal enteric flora if campylobacter species of vibrio and parasite oxytoca present. patient symptoms warrant campylobacter antigen result positive flora isolated

APPENDIX C
List of top 20 features selected from the real
and synthetic datasets using gini impurity scores.

| Rank | Real (train) dataset | Synthetic dataset |
|---|---|---|
| 1 | Shigella | Salmonella |
| 2 | Salmonella | Speci |
| 3 | Speci | Shigella |
| 4 | Isol | Health |
| 5 | Campylobact | Campylobact |
| 6 | Health | Isol |
| 7 | Indiana | Sct |
| 8 | Group | State |
| 9 | Suscept | Group |
| 10 | Typhi | Confirm |
| 11 | Confirm | Chslb |
| 12 | Depart | Indiana |
| 13 | MI | Suscept |
| 14 | Spp | Typhi |
| 15 | Call | Call |
| 16 | Cultur | Depart |
| 17 | Stool | Sent |
| 18 | Chslb | Test |
| 19 | Coli | Self |
| 20 | Enter | Cultur |

APPENDIX D
Intersection of top 5, 10, 15, 20, 50 and 100 features selected
from the real and synthetic datasets using gini impurity scores.

| Feature | # features | |
|---|---|---|
| subset | present in both | |
| size | datasets | List of features present in both datasets |
| 5 | 4 (80%) | salmonella, speci, shigella, campylobact |
| 10 | 7 (70%) | speci, health, isol, group, salmonella, shigella, campylobact |
| 15 | 12 (80%) | speci, indiana, campylobact, confirm, health, isol, group, |
| | | suscept, typhi, salmonella, shigella, call |
| 20 | 14 (70%) | chslb, speci, indiana, shigella, campylobact, confirm, |
| | | health, isol, group, suscept, depart, salmonella, typhi, call |
| 50 | 35 (70%) | sct, speci, non, due, isol, suscept, typhi, coli, report, chslb, |
| | | call, indiana, cultur, confirm, diseas, issu, gener, |
| | | azithromycin, sent, health, gastroenter, without, self, |
| | | progress, depart, salmonella, stool, campylobact, enter, |
| | | medic, test, final, group, shigella, tofmass |
| 100 | 79 (79%) | perform, sct, present, speci, characterist, non, laboratori, |
| | | due, isol, result, pathogen, infect, suscept, typhi, coli, |
| | | identifi, report, chslb, serogroup, call, indiana, cultur, thi, lab, |
| | | confirm, enzym, sourc, aerob, diseas, growth, issu, drug, gener, |
| | | azithromycin, moder, maldi, specimen, sent, gastroenter, |
| | | health, without, underli, serotyp, spectrometri, routin, self, |
| | | toxin, numer, aeromona, follow, salmonella, progress, |
| | | depart, stool, tofmass, ser, enter, campylobact, normal, |
| | | develop, shiga, medic, patient, salsp, determin, test, mani, |
| | | choic, mass, final, group, usual, see, board, date, shigella, access |

### APPENDIX F. PRESENCE DISCLOSURE TEST

A frequency of a synthetic report is computed matching with 1-to-n many real reports using hamming score thresholds of 10 and 20 (10=reports are more similar, 20=reports are less similar). It can be hypothesized that negative synthetic reports stood a much greater chance of matching with negative (train) reports because negative (train) reports tend to be similar to each other due to uniform text used to report a negative outcome. Thus, separate tests were performed against positive and negative matched to each synthetic report. Next, the frequency of n synthetic reports matching with m training reports can be plotted.

We claim:

1. A method of generating synthetic medical data for enabling machine learning research, comprising:
leveraging two adversarial neural networks that compete with each other to create a synthetic message dataset that substantially mimics real medical data; and
lowering re-identification risk associated with the synthetic message dataset based on presence disclosure assessment, wherein the synthetic message dataset is compared to the real medical data using hamming distance thresholds.

2. The method of claim 1, further comprising generating a positive train dataset using the two adversarial networks.

3. The method of claim 2, further comprising generating a positive holdout dataset using the two adversarial networks, wherein the positive holdout dataset excludes the positive train dataset.

4. The method of claim 3, wherein the synthetic message dataset is used to train a classification model, wherein the classification model is tested using the positive holdout dataset.

5. The method of claim 1, further comprising generating a negative train dataset using the two adversarial networks.

6. The method of claim 4, further comprising generating a negative holdout dataset using the two adversarial networks, wherein the negative holdout dataset excludes the negative train dataset.

7. The method of claim 6, wherein the synthetic message dataset is used to train a classification model, wherein the classification model is tested using the negative holdout datasets.

8. The method of claim 1, wherein the lowering the re-identification risk associated with the synthetic message dataset involves a presence disclosure test that compares synthetic data records with real data records using the hamming distance thresholds.

9. The method of claim 8, further comprising determining a degree of the re-identification risk based on the hamming distance thresholds.

10. A method of processing real medical data, comprising:
generating a first message dataset from the real medical data using a first neural network;
generating a second message dataset from the real medical data using a second neural network;
generating a synthetic message dataset having at least a portion of the medical data based on the first message dataset and the second message dataset, the synthetic message dataset having synthetic medical data being substantially indistinguishable from the real medical data; and
lowering a re-identification risk associated with the synthetic message dataset based on a match between a synthetic data record in the synthetic medical data and a real data record in the real medical data.

11. The method of claim 10, wherein generating the first message comprises generating a positive train dataset of the first message dataset for training the first neural network.

12. The method of claim 11, wherein generating the first message comprises generating a positive holdout dataset of the first message dataset that excludes the positive train dataset.

13. The method of claim 11, wherein generating the synthetic message dataset comprises generating a positive model based on the positive train dataset.

14. The method of claim 10, wherein generating the second message comprises generating a negative train dataset of the second message dataset for training the second neural network.

15. The method of claim 14, wherein generating the second message comprises generating a negative holdout dataset of the first message dataset that excludes the negative train dataset.

16. The method of claim 14, wherein generating the synthetic message dataset comprises generating a negative model based on the negative train dataset.

17. The method of claim 10, wherein lowering the re-identification risk associated with the synthetic message dataset comprises using a hamming distance between the synthetic data record and the real data record.

18. The method of claim 17, further comprising determining a degree of the re-identification risk based on the hamming distance.
